(19) United States
(12) Patent Application Publication
(10) Pub. No.: US 2016/0175426 A1
(43) Pub. Date: Jun. 23, 2016

(54) COMPOSITIONS FOR MUCUSAL DELIVERY, USEFUL FOR TREATING PAPILLOMAVIRUS INFECTIONS

(71) Applicant: UNIVERSITY OF LOUISVILLE RESEARCH FOUNDATION, INC., Louisville, KY (US)

(72) Inventors: Kenneth E. Palmer, Owensboro, KY (US); Nobuyuki Matoba, Owensboro, KY (US); Joshua Fuqua, Owensboro, KY (US)

(21) Appl. No.: 14/905,308

(22) PCT Filed: Jul. 16, 2014
(86) PCT No.: PCT/US14/46893

§ 371 Date: Jan. 15, 2016

Related U.S. Application Data
(60) Provisional application No. 61/846,906, filed on Jul. 16, 2013.

(51) Int. Cl. A61K 39/12; C07K 14/28; C12N 7/00; C07K 14/005

(52) U.S. Cl. CPC A61K 39/12; C07K 14/005; C07K 14/28; C12N 7/00; A61K 2039/541

(57) ABSTRACT

Polypeptides, compositions, and methods for treatment of papillomavirus (PV) infections including a papillomavirus (PV) minor capsid (L2) polypeptide fragment and a cholera toxin B subunit (CTB) polypeptide are described. Polypeptides and compositions disclosed herein can be administered to the mucosa of a subject, resulting in unexpectedly beneficial production of cross-neutralizing antibodies.

### RELATED APPLICATIONS

This application claims priority from U.S. Provisional Application Ser. No. 61/846,906 filed Jul. 16, 2013, the entire disclosure of which is incorporated herein by this reference.

### GOVERNMENT INTEREST

This invention was made with government support under W81XWH-10-2-0082 CLIN2 and CLIN 3 awarded by the Department of Defense. The government has certain rights in the invention.

### TECHNICAL FIELD

The presently-disclosed subject matter relates to polypeptides, compositions, and methods for treatment of papillomavirus (PV) infections. In particular, the presently-disclosed subject matter relates to polypeptides, compositions, and methods that provide mucosal delivery and a spectrum of protection against multiple PV types.

### INTRODUCTION

Papillomaviruses are species-specific, anatomic-site restricted small DNA tumor viruses that cause a variety of pathologies of differing degrees of severity. Certain types of papillomaviruses (PVs) can cause papillomas or warts. These warts can occur in a variety of locations on an infected subject. Warts caused by PV are sometimes found in the genital region of an infected subject. In some cases, these warts can infect babies born to infected mothers. In such a situation, the child can develop recurrent respiratory papillomatosis (RRP), where papillomas develop in the respiratory tract. These respiratory papillomas can be deadly in pediatric RRP due to the small size of the upper airway in children. Papillomaviruses (PVs) are also associated with up to 99% of cervical cancers and more than 25% of oropharyngeal cancers. Through use of cervical screening tests, the incidence of invasive cervical cancers in developed countries has decreased, but still occurs in a number of women in developed countries. In developing and underdeveloped countries invasive cervical cancer is an even greater threat.

Human papillomaviruses (HPVs) associated with genital warts include HPV-6 and HPV-11. HPVs implicated in the etiology of cervical cancer include: HPV-16, HPV-18, HPV-31; HPV-33; HPV-35; HPV-39; HPV-45, HPV-52, HPV-58, and HPV-68. Research in the past decade has generated a wealth of knowledge on the correlates of protection against papillomavirus infection. Investigators have attempted to develop compositions to prevent infection with some of the HPV-types known to cause cervical cancer, anogenital cancer, head and neck cancers, other mucosal cancer, and genital warts.

HPVs have two outer coat proteins, the major capsid protein (L1; about 85% of coat protein) and the minor capsid protein (L2; about 15% of coat protein). A composition effective against certain HPV-types has been produced using virus-like particles (VLPs) of the L1 major capsid protein. Preclinical studies were conducted in a canine oral papillomavirus (COPV), bovine papillomavirus type 4 (BPV-4) and cottontail rabbit papillomavirus (CRPV) systems, which are established as the models-of-choice for use in preclinical studies in the field. Preclinical studies were 100% successful, and clinical studies in humans were 100% successful as well, further establishing the efficacy of the L1-VLP composition. Uninfected women who were vaccinated with the VLPs comprising the L1 major capsid protein of HPV-16 were protected against acquisition of chronic HPV-16 infection and development of cervical intraepithelial neoplasia (CIN), e.g., CIN-2, which is the premalignant lesion selected as the endpoint for clinical trials against development of cervical cancer.

Although the L1 VLPs were successful, existing treatment options for HPV infection still suffer from certain drawbacks. For example, the breadth of antigenic diversity present in this group of pathogens makes induction of broadly neutralizing antibodies through current modes of treatment very difficult. L1-based compositions do not appear capable of inducing antibodies with neutralizing activities functional against a broad range of papillomavirus types. In this regard, while known L1 VLPs for treating HPV infection have high likelihood of protecting women against infection with, two, three, or even four different types of HPV, where multi-valent compositions are provided, they may not protect against infection with other types. Indeed, clinical data obtained during studies related to the efficacy of the L1 VLPs show that in control and vaccinated test groups, approximately the same number of subjects in each group developed CIN associated with HPV infection from types other than HPV-16 infection.

On the other hand, studies have indicated that the minor capsid protein (L2) appears to have some ability to induce cross-neutralizing antibodies. (See Campo (1997) Clin Dermatol; Campo (1997) Virology; Kawana (2001) Vaccine; Kawana (1998) Virology; Kawana (2003) Vaccine; Kawana (1999) J Virol; Kawana (2001) J Virol; Slupetzky (2007) Vaccine; Varsani (2003) J Virol; Gambhira (2006) Cancer Res; Gambhira (2007) J Virol; Gambhira (2007) J Virol; Kim (2008) Vaccine; Pastrana (2005) Virology; Roden (2000) Virology). Despite promising results from the perspective that cross-neutralizing antibodies could be induced, attempts at making L2-based treatment compositions were not entirely successful. They were found to be poorly immunogenic and neutralizing titers induced on administration were low. Consequently, the outcome of treatment was highly variable. (See Id).

Also among the drawbacks of existing HPV treatment options is their associated cost. For example, production of the L1 proteins in cultured cell systems, particularly those requiring use of fermentation, is expensive, e.g., using viral expression systems, such as, baculovirus expression system, a yeast expression system, or bacterial expression systems, such as an E. coli expression system. The cost of L1-VLPs currently on the market is approximately $360 for three doses, making them available to only the portion of the population in developed countries able to absorb such health-care costs, and very little, if any, of the population in developing or underdeveloped countries having few economic resources.

Also among the drawbacks of existing HPV treatment options are their limited routes of administration.

Accordingly, there remains a need in the art for compositions and methods for treating PV infections that address the above-identified drawbacks of existing treatment options.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter polypeptides, compositions including such polypeptides, nucleotides, expression vectors, plant cells, and methods for use thereof. The disclosed subject matter is related to the treatment of papillomavirus (PV) infections. Such treatment can make use of the polypeptides and compositions disclosed herein, which include a papillomavirus (PV) minor capsid (L2) polypeptide fragment and a cholera toxin B subunit (CTB) polypeptide. Polypeptides and compositions disclosed herein can be administered to the mucosa of a subject, resulting in unexpectedly beneficial production of cross-neutralizing antibodies. In this regard, in some embodiments, the polypeptide or composition comprises an HPV L2 polypeptide fragment from a first HPV-type, and the polypeptide or composition is effective for treatment of infections caused by the first HPV-type and at least one additional HPV-type.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A shows the amino acid sequence of the human papillomavirus (HPV)-16 minor capsid (L2) protein (SEQ ID NO: 1), in which the RG-1 epitope is italicized, the 56-75 epitope is [symbol] , the 8-37 fragment is d̅o̅u̅b̅l̅e̅ u̅n̅d̅e̅r̅l̅i̅n̅e̅d̅, the 1-90 fragment is [symbol] [symbol] , , ↓ identifies the furin cleavage site, and the Kawana epitope is single underlined.

FIG. 1B shows the amino acid sequence of the cholera toxin B subunit (CTB) (SEQ ID NO: 3), in which the histidine at residue 13 is underlined, which histidine can be replaced with an arginine in some embodiments of the polypeptide disclosed herein.

FIG. 1C depict exemplary embodiments of polypeptides provided in accordance with the presently-disclosed subject matter, which include an 8-37 HPV-16 L2 fragment that contains the RG-1 epitope fused to the N-terminal or C-terminal of CTB, and a 6×His tag was added to the same terminus as the HPV epitope to aid in purification.

FIG. 2 depicts the results of studies to assess antigen stability through western blots and SDS-PAGE to confirm the presence of CTB and the L2 epitope.

FIG. 3. depicts the results of a series of HPV Pseudovirus Neutralization Assays and L2 Serology studies. Neutralization titers against HPV-16, 18, 31, and 45 were assessed in serum from two rabbits inoculated with C-CTB-L2 and N-CTB-L2. The inverse of the final dilution exhibiting 50% virus inhibition was averaged between animals. Serum from animals vaccinated with N-CTB-L2 appear to cross-neutralize virus more effectively. Antibodies reactive against L2 were assessed by ELISA using a BSA conjugated peptide homologous to the RG-1 region of HPV-16 L2 immobilized on the plate. Endpoint titers were determined to be the last dilution with a signal above 2× the background signal.

FIG. 4 depicts the results of IMAC purification of CTB-L2.

FIG. 5 depicts the results of studies to assess cellular GM-1 binding of antigens. Interactions of CTB-L2 with the surface of cells through GM-1 binding was investigated through double fluorescence of CTB and the L2 region while competing with soluble GM-1. These data indicate that both N-CTB-L2 and C-CTB-L2 can bind to immune effector cells throught GM-1. Analyzed by a two-way ANOVA with Bonferroni's post-hoc tests: N-CTB-L2 vs. CTB—̂p<0.05, ̂̂p<0.01, C-CTB-L2 vs. CTB— [symbol] p< [symbol] p<0.001, N-CTB-L2 vs. C-CTB-L2—* p<0.05.

FIGS. 6A-6I depict the results of a series of HPV Pseudovirus Neutralization Assays and L2 Serology studies. Neutralization titers against HPV-16, 18, 31, 45, 58, 6, and 11, and COPV were assessed in serum from two rabbits inoculated with C-CTB-L2, two rabbits inoculated with N-CTB-L2, and two rabbits inoculated with Gardasil®. Serum from animals vaccinated with N-CTB-L2 appears to cross-neutralize virus most effectively.

FIGS. 7A and 7B depict the results of a mouse mucosal immunization trial. Neutralization titers were averaged across each group and analyzed by a two-way ANOVA with Bonferroni's post-hoc tests. No significant differences were observed in HPV 16 neutralizations of C-CTB-L2 or N-CTB-L2 vaccinated animals when comparing the same route of administration across vaccine candidates. Significant differences were observed when comparing HPV 16 neutralizations across modes of delivery within each treatment group. Delivery produced significantly less neutralizing antibodies when compared to sublingual delivery for both constructs. Serum from animals inoculated with N-CTB-L2 demonstrated significantly greater cross-neutralization of HPV18 when delivered sublingually relative to C-CTB-L2 delivered sublingually or N-CTB-L2 delivered orally or subcutaneously. n=5 ** p-value <0.01., **** p-value <0.0001.

FIG. 8 depicts the results of a mouse mucosal immunization trial, showing delivery of CTB-L2 via mucosal routes provides immune response to produce cross-neutralizing HPV antibodies for HPV-16, 18, 31, 45, and 58. Analyzed by a two-way ANOVA with Bonferroni's post-hoc tests. No significant differences. n=4.

### BRIEF DESCRIPTION OF THE SEQUENCE LISTING

SEQ ID NO: 1 is the amino acid sequence of the human papillomavirus (HPV)-16 minor capsid (L2) protein;

SEQ ID NO: 2 is a cDNA sequence encoding the polypeptide of SEQ ID NO:1.

SEQ ID NO: 3 is the amino acid sequence of the cholera toxin B subunit (CTB);

SEQ ID NO: 4 is the amino acid sequence of the cholera toxin B subunit (CTB), wherein the histidine at residue 13 has been replaced with an arginine;

SEQ ID NO: 5 is a cDNA sequence encoding the polypeptide of SEQ ID NO: 4;

SEQ ID NO: 6 is the amino acid sequence of a polypeptide provided in accordance with the present invention, which includes an HPV polypeptide fragment adjacent the amino-terminal end of a CTB (N-CTB-L2);

SEQ ID NO: 7 is a cDNA sequence encoding the polypeptide of SEQ ID NO: 6;

SEQ ID NO: 8 is the amino acid sequence of another polypeptide provided in accordance with the present invention, which includes an HPV polypeptide fragment adjacent the carboxy-terminal end of a CTB (C-CTB-L2);

SEQ ID NO: 9 is a cDNA sequence encoding the polypeptide of SEQ ID NO: 8.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter polypeptides, compositions including such polypeptides, nucleotides, expression vectors, plant cells, and methods for use thereof. The disclosed subject matter is related to the treatment of papillomavirus (PV) infections. Such treatment can make use of the polypeptides and compositions disclosed herein, which include a papillomavirus (PV) minor capsid (L2) polypeptide fragment and a cholera toxin B subunit (CTB) polypeptide. Polypeptides and compositions disclosed herein can be administered to the mucosa of a subject, resulting in unexpectedly beneficial production of cross-neutralizing antibodies. In this regard, in some embodiments, the polypeptide or composition comprises an HPV L2 polypeptide fragment from a first HPV-type, and the polypeptide or composition is effective for treatment of infections caused by the first HPV-type and at least one additional HPV-type.

As used herein, a “PV L2 polypeptide fragment” (sometimes referred to herein as PV L2 polypeptide) is an isolated polypeptide comprising the amino acid sequence of a fragment of a full-length PV minor-capsid (L2) protein (including PV L2 “consensus” polypeptides). In some embodiments, a PV L2 polypeptide can be a human papillomavirus (HPV) L2 polypeptide. In some embodiments, a PV L2 polypeptide can be a canine oral papillomavirus (COPV) L2 polypeptide.

An HPV L2 polypeptide fragment is an isolated polypeptide comprising the amino acid sequence of a fragment of a full-length HPV minor-capsid (L2) protein (including HPV L2 consensus polypeptides). An HPV L2 polypeptide can be provided from any HPV-type. Examples of HPV-types include, but are not limited to, HPV-6, HPV-11, HPV-16, HPV-18, HPV-26, HPV-31, HPV-33, HPV-35, HPV-39, HPV-40, HPV-45, HPV-51, HPV-52, HPV-53, HPV-56, HPV-58, HPV-59, HPV-68, HPV-73, and HPV-82. HPV-types can further include subtypes; for example, when HPV-16 is referenced herein, it is understood to refer to HPV-16a and HPV-16b, and other subtypes that could be discovered. The full length amino acid sequence of HPV-16 is provided as SEQ ID NO: 1. As will be understood by those of ordinary skill in the art, the amino acid and nucleotide sequences of PVs are available, for example, in the UniProt Knowledgebase, and can be accessed by searching by name or by SwissProt accession number. SwissProt accession numbers for some HPVs are as follows, and others can be easily obtained by those of ordinary skill in the art: HPV 6 L2 (Q84297); HPV 11 L2 (P04013); HPV-18 L2 (P06793); HPV-31 L2 (P17389); HPV-33 L2 (P06418); HPV 35 L2 (P27234); HPV-45 L2 (P36761); HPV 52 L2 (P36763); and HPV 58 L2 (P26538).

In some instances, it can be useful to describe papillomaviruses as being grouped into categories. For example, papillomaviruses can be grouped into “genera,” as set forth in de Villiers, et al., (2004) Classification of papillomavirus, Virology 324:1, pp. 17-27, which is incorporated herein by this reference. Within each genus of de Villiers, et al., are a group of so-called “species.” Each papillomavirus type can be described as being within a particular species. For example, genus alpha-papillomavirus, species 9, includes the following HPV-types: HPV-16, -31, -33, -35, -52, -58, and -67. For another example, genus alpha-papillomavirus, species 7, includes the following HPV-types: HPV-18, -45, -49, -68, and -70. For yet another example, genus alpha-papillomavirus, species 10, includes the following HPV-types: HPV-6, -11, and -13.

The term “isolated”, when used in the context of an isolated polynucleotide, isolated polypeptide, or isolated antibody is a polynucleotide, polypeptide, or antibody that, by the hand of man, exists apart from its native environment and is therefore not a product of nature. An isolated polynucleotide or polypeptide can exist in a purified form or can exist in a non-native environment such as, for example, in a transgenic host cell.

The term “native” or “wild type” refers to a gene that is naturally present in the genome of an untransformed cell. Similarly, when used in the context of a polypeptide, “native” or “wild type” refers to a polypeptide that is encoded by a native gene of an untransformed cell's genome.

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment,” when used in reference to a polypeptide, refers to a polypeptide in which amino acid residues are deleted as compared to the reference polypeptide itself, but where the remaining amino acid sequence is usually identical to the corresponding positions in the reference polypeptide, such as for example a native polypeptide. Such deletions can occur at the amino-terminus or carboxy-terminus of the reference polypeptide, or alternatively both. Fragments typically are at least about 20, 30, 40, 50, 60, 70, 80, 90, or 100 amino acids long. In some embodiments of the presently-disclosed subject matter, the fragments primarily include residues from the amino-terminal region of a PV L2 polypeptide.

A fragment can also be a “functional fragment,” in which case the fragment is capable of affecting treatment of a PV infection. In some embodiments, a functional fragment of a reference (e.g., native) polypeptide retains some or all of the ability of the reference polypeptide to affect treatment of a PV infection. In some embodiments, a functional fragment of a reference polypeptide has an enhanced ability, relative to the reference polypeptide, to affect treatment of a PV infection. In some embodiments, the reference polypeptide is a full-length native HPV L2 protein.

The term “variant” refers to an amino acid sequence that is different from the reference (e.g., native) polypeptide by one or more amino acids, e.g., one or more amino acid substitutions. A variant of a reference polypeptide also refers to a variant of a fragment of the reference polypeptide, for example, a fragment wherein one or more amino acid substitutions have been made relative to the reference polypeptide. A variant can also be a “functional variant,” in which case the fragment is capable of affecting treatment of a PV infection. In some embodiments, a functional variant of a reference polypeptide retains some or all of the ability of the reference polypeptide to affect treatment of a PV infection. In some embodiments, a functional variant of a reference polypeptide has an enhanced ability, relative to the reference polypeptide, to affect treatment of a PV infection. In some embodiments, the reference polypeptide is a full-length native HPV L2 protein.

The term functional variant further includes conservatively substituted variants. The term “conservatively substituted variant” refers to a polypeptide comprising an amino acid residue sequence that differs from a reference polypeptide by one or more conservative amino acid substitutions, and is capable of affecting treatment of a PV infection. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; the substitution of one acidic residue, such as aspartic acid or glutamic acid for another; or the substitution of one aromatic residue, such as phenylalanine, tyrosine, or tryptophan for another. The phrase “conservatively substituted variant” also includes polypeptides wherein a residue is replaced with a chemically derivatized residue, provided that the resulting polypeptide is capable of affecting treatment of a PV infection.

In some embodiments, the PV L2 polypeptide can be a polypeptide having 85, 86, 87, 88, 89, 90, 91, 92, 93, 94, 95, 96, 97, 98, or 99% homology to the amino acid sequence of a full-length native PV minor-capsid (L2) protein, or a functional fragment thereof, so long as the resulting PV L2 polypeptide is capable of affecting treatment of a PV infection. In some embodiments, the PV L2 polypeptide fragment is a polypeptide having 85, 86, 87, 88, 89, 90, 91, 92, 93, 94, 95, 96, 97, 98, or 99% homology to the amino acid sequence of a fragment of wild type HPV-16 L2 polypeptide.

“Percent similarity” and “percent homology” are synonymous as herein and can be determined, for example, by comparing sequence information using the GAP computer program, available from the University of Wisconsin Geneticist Computer Group. The GAP program utilizes the alignment method of Needleman et al. (1970) J. Mol. Biol. 48:443, as revised by Smith et al. (1981) Adv. Appl. Math. 2:482. Briefly, the GAP program defines similarity as the number of aligned symbols (i.e. nucleotides or amino acids) which are similar, divided by the total number of symbols in the shorter of the two sequences. The preferred default parameters for the GAP program include: (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps. The term “homology” describes a mathematically based comparison of sequence similarities which is used to identify genes or proteins with similar functions or motifs. Accordingly, the term “homology” is synonymous with the term “similarity” and “percent similarity” as defined above. Thus, the phrases “substantial homology” or “substantial similarity” have similar meanings.

As noted above, polypeptides and compositions of the presently-disclosed subject matter include a PV L2 polypeptide fragment, which can comprise a fragment of a PV minor-capsid (L2) protein. In some embodiments, the PV L2 polypeptide has the sequence of a HPV L2 polypeptide fragment. In some embodiments, the HPV L2 polypeptide fragment is from an HPV-type, selected from the group consisting of HPV-1, -2, -5, -6, -8, -6, -11, -16, -18, -26, -31, -33, -35, -39, -40, -45, -51, -52, -53, -56, -58, 59, -67, -68, -73, and -82. In some embodiments, the HPV L2 polypeptide fragment is from an HPV-type, selected from the group consisting of HPV-16, 31, 33, 35, 52, 58, and 67. In some embodiments, the HPV L2 polypeptide fragment is from an HPV-type, selected from the group consisting of HPV-18, 45, 49, 68, and 70. In some embodiments, the HPV L2 polypeptide fragment is from an HPV-type, selected from the group consisting of HPV-6, 11, and 13. In some embodiments, the HPV L2 polypeptide fragment is an HPV-16 L2 polypeptide fragment.

Such polypeptides and compositions of the presently-disclosed subject matter induce production of cross-neutralizing antibodies when administered to a mucosa of a subject. In some embodiments, such polypeptides and compositions induce production of cross-neutralizing antibodies when administered sublingually to a subject. For example, administration of a polypeptide or composition including a fragment of a HPV minor-capsid (L2) protein could induce production of cross-neutralizing antibodies to multiple HPV-types, including but not limited to, HPV-1, -2, -5, -6, -8, -6, -11, -16, -18, -26, -31, -33, -35, -39, -40, -45, -51, -52, -53, -56, -58, 59, -67, -68, -73, and/or -82

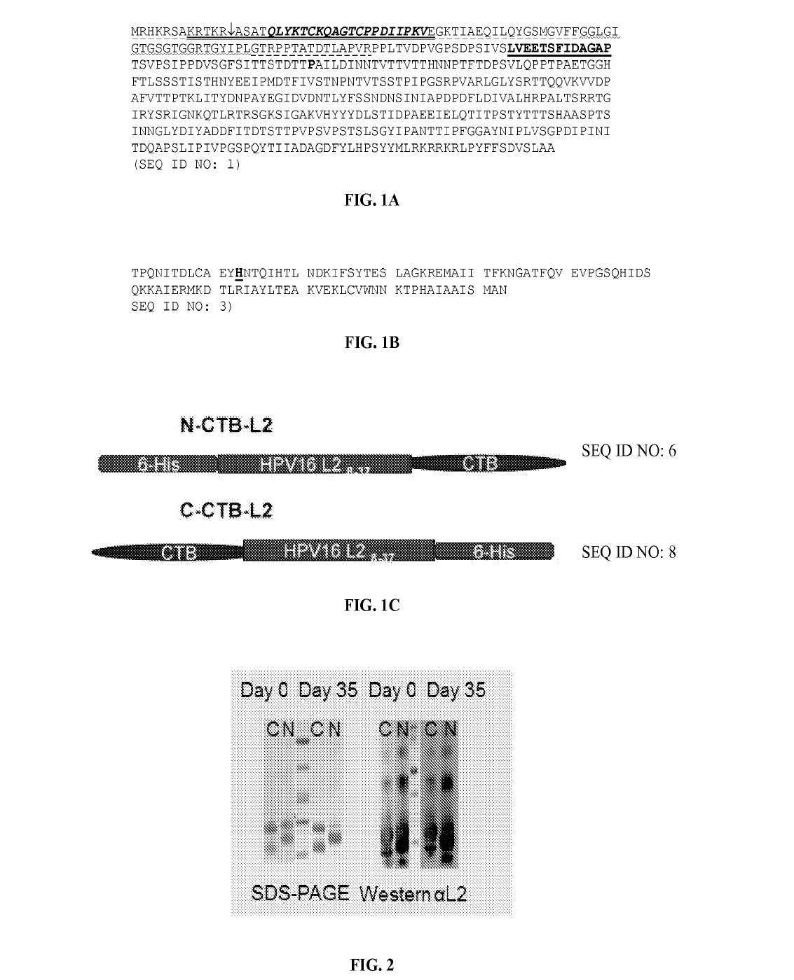

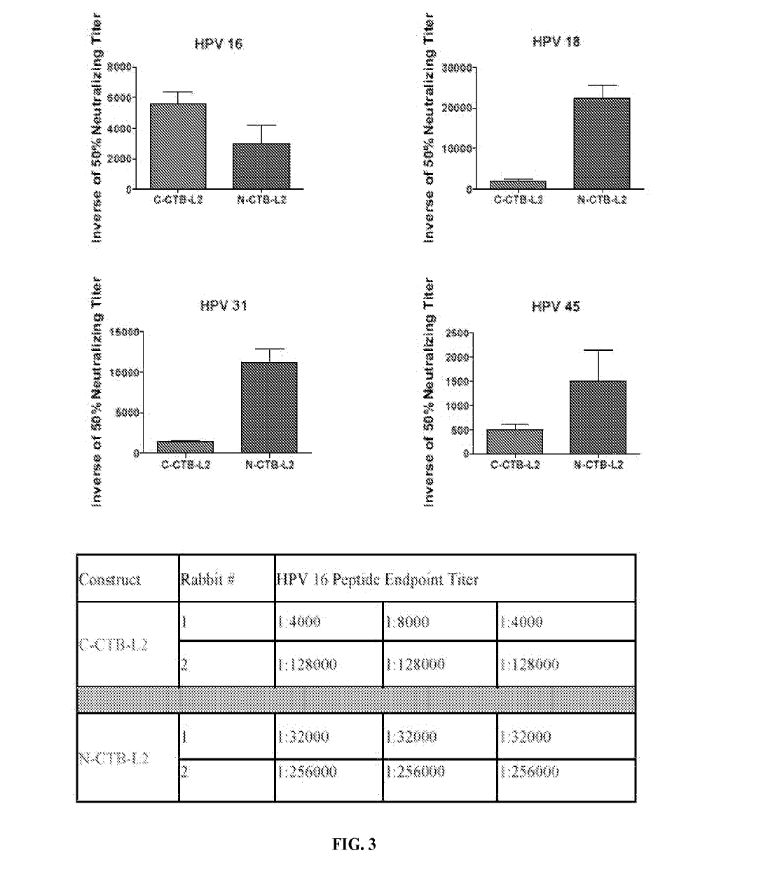

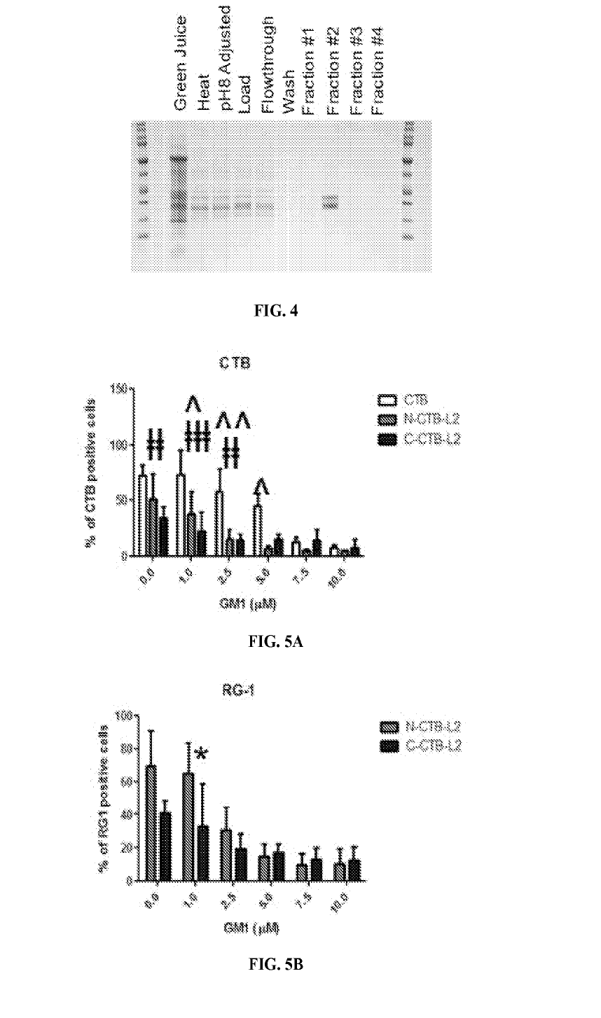

Unless otherwise specified, a reference to a PV fragment is not specific for a particular PV or PV-type. In this regard, HPV polypeptides of different types (and even other PVs, e.g., COPV) are highly conserved, particularly in the amino-terminal portion of the polypeptides, extending from about amino acid 1 to about amino acid 260. As such, for example, a fragment extending from amino acid 5, 6, 7, 8, 9, 10, 11, 12, 13, 14, 15, 16, 17, or 18, and extending to amino acid 120, 115, 110, 105, 100, 95, 90, 85, 80, 75, 74, 73, 72, 71, 70, 69, 68, 67, 66, or 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, or 35 can be of interest in PVs of different types. In some embodiments, a fragment extending from Furin cleavage and including the Kawana epitope in COPV and HPV of different types can be of interest. In some embodiments, a fragment within residues 1-100 fragment can be of interest in COPV and HPV of different types. Using HPV-16 as an initial example, if a 13-120, 1-90, 8-37, or 17-36 fragment of HPV-16 (identified in FIG. 1) is selected as a fragment of interest, the same region in any other alpha papillomavirus HPV L2 sequence or COPV sequence can be selected as a polypeptide of interest.

Indeed, the identified residues of 13-120 in HPV-16, for example, would translate into the other PV types, with additions or subtractions of less than fifteen, less than ten, less than five, less than two, or no residues. For example, the 13-120 region of HPV-16 is equivalent to the 13-120 region of HPV6, HPV-11, HPV-26, HPV-31, and HPV-58. For another example, the 13-120 region of HPV-16 is equivalent to the 13-119 region of HPV-18 because HPV-18 has one fewer amino acid in the region. As such, when the 13-120 region of HPV-16 is of interest, the 13-119 region of HPV-18 could be of interest as well. Nevertheless, the conservation in the amino-terminal portion is such that the 13-120 region of HPV-18 could also be of interest. For another example, the 13-120 region of HPV-16 is equivalent to the 13-132 region of COPV because COPV includes 13 additional amino acids in the region. Thus, when the 13-120 region of HPV-16 is of interest, the 13-132 region of COPV could also be of interest. Nevertheless, the conservation in the amino-terminal portion is such that the 13-120 region of COPV could also be of interest. As such, as used herein, when a PV fragment is identified, the fragment should not be considered PV-type-specific, and can be of interest in an HPV L2 sequence, unless otherwise specified.

In some embodiments, the composition comprises a PV L2 polypeptide comprising a fragment. In some embodiments, the fragment can include of amino acids from the amino-terminal portion of a PV minor capsid (L2) protein, for example, amino acids from the portion extending from about amino acid 1 to about amino acid 260. In some embodiments, the fragment can begin at (i.e., extend from) about amino acid 1, 2, 3, 4, 5, 6, 7, 8, 9, 10, 11, 12, 13, 14, 15, 16, 17, 18, 19, or 20. In some embodiments, the functional fragment can end at (i.e., extend to) about amino acid 120, 115, 110, 105, 100, 95, 90, 85, 80, 75, 74, 73, 72, 71, 70, 69, 68, 67, 66, or 65.

In some embodiments, the PV L2 polypeptide fragment comprises a PV L2 fragment or an HPV L2 fragment selected from: 5-120, 5-100, 5-90, 5-75, 5-70, 5-65, 5-60, 5-55, 5-50, 5-45, 5-40, 5-39, 5-38, 5-37, 5-36, 5-35, 5-34, 5-33, 5-32, 5-31, 5-30, 6-120, 6-100, 6-90, 6-75, 6-70, 6-65, 6-60, 6-55, 6-50, 6-45, 6-40, 6-39, 6-38, 6-37, 6-36, 6-35, 6-34, 6-33, 6-32, 6-31, 6-30, 7-120, 7-100, 7-90, 7-75, 7-70, 7-65, 7-60, 7-55, 7-50, 7-45, 7-40, 7-39, 7-38, 7-37, 7-36, 7-35, 7-34, 7-33, 7-32, 7-31, 7-30, 8-120, 5-100, 5-90, 5-75, 5-70, 8-65, 8-60, 8-55, 8-50, 8-45, 8-40, 8-39, 8-38, 8-37, 8-36, 8-35, 8-34, 8-33, 8-32, 8-31, 8-30, 9-120, 9-100, 9-90, 9-75, 9-70, 9-65, 9-60, 9-55, 9-50, 9-45, 9-40, 9-39, 9-38, 9-37, 9-36, 9-35, 9-34, 9-33, 9-32, 9-31, 9-30, 10-120, 10-100, 10-90, 10-75, 10-70, 10-65, 10-60, 10-55, 10-50, 10-45, 10-40, 10-39, 10-38, 10-37, 10-36, 10-35, 10-34, 10-33, 10-32, 10-31, 10-30, 11-120, 11-100, 11-90, 11-75, 11-70, 11-65, 11-60, 11-55, 11-50, 11-45, 11-40, 11-39, 11-38, 11-37, 11-36, 11-35, 11-34, 11-33, 11-32, 11-31, 11-30, 12-120, 12-100, 12-90, 12-75, 12-70, 12-65, 12-60, 12-55, 12-50, 12-45, 12-40, 12-39, 12-38, 12-37, 12-36, 12-35, 12-34, 12-33, 12-32, 12-31, 12-30, 13-120, 13-100, 13-90, 13-75, 13-70, 13-65, 13-60, 13-55, 13-50, 13-45, 13-40, 13-39, 13-38, 13-37, 13-36, 13-35, 13-34, 13-33, 13-32, 13-31, 13-30, 14-120, 5-100, 5-90, 5-75, 5-70, 14-65, 14-60, 14-55, 14-50, 14-45, 14-40, 14-39, 14-38, 14-37, 14-36, 14-35, 14-34, 14-33, 14-32, 14-31, and 14-30.

In some embodiments, the composition comprises a PV L2 polypeptide comprising or consisting essentially of a fragment that begins at about the amino acid adjacent and downstream a furin cleavage site (See Richards, et al., (2006) PNAS, identifying the furin cleavage site of L2, incorporated herein by reference). The furin cleavage site of L2 is close to the amino-terminus, and follows a motif including the amino acids RXKR, where X is any amino acid.

The L2 furin cleavage site for HPV-16, HPV-31, and HPV-35, for example, is between amino acids 12 and 13. As such, in some embodiments, the functional fragment is from HPV-16, HPV-31, or HPV-35, or a functional variant thereof, and begins at about amino acid 13. For another example, the L2 furin cleavage site for HPV-6, HPV-18, HPV-26, HPV-40, HPV-45, HPV-53, and HPV-58 is between amino acids 11 and 12. As such, in some embodiments, the functional fragment, or a functional variant thereof, is from HPV-6, HPV-18, HPV-26, HPV-40, HPV-45, HPV-53, or HPV-58 and begins at about amino acid 12. For another example, the L2 furin cleavage site for HPV-11 is between amino acids 10 and 11. As such, in some embodiments, the fragment, or a functional variant thereof, is from HPV-11 and begins at about amino acid 11.

In some embodiments, a composition of the presently-disclosed subject matter can include a PV L2 polypeptide capable of inducing antibodies with neutralizing activities functional against a broad range of papillomavirus types. In this regard, a cross-neutralization or cross-neutralizing activity refers to the ability of a PV L2 polypeptide from a first primary PV-type to affect treatment of infections caused by the primary PV-type and at least one additional secondary PV-type.

In some embodiments, the consensus PV L2 polypeptide can comprise a variant fragment of a native PV L2 polypeptide. The fragment can be a PV L2 fragment as disclosed herein, or as known in the art. In some embodiments, for example, the consensus PV L2 polypeptide is a variant fragment of a primary PV-type L2 native sequence having an amino acid sequence extending from a furin cleavage site to a downstream amino acid between about 65 and about 260 of the primary PV L2 native sequence. In some embodiments, the consensus PV L2 polypeptide is a variant fragment of a primary PV-type L2 native sequence having an amino acid sequence extending from amino acid 3, 4, 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of the primary PV L2 native sequence.

As noted above, polypeptides and compositions of the presently-disclosed subject matter include a PV L2 polypeptide fragment and a cholera toxin B subunit (CTB) polypeptide.

In some embodiments, the CTB polypeptide has the sequence of the polypeptide of SEQ ID NO: 3. In some embodiments, the CTB polypeptide has the sequence of the polypeptide of SEQ ID NO: 4. In some embodiments, the CTB polypeptide has the sequence of a fragment of the polypeptide of SEQ ID NO: 3 or 4. In some embodiments, the CTB polypeptide has the sequence of a fragment of the polypeptide of SEQ ID NO: 3 or 4, extending from amino acid 2, 3, 4, 5, 6, or 7, and extending to amino acid 103, 102, 101, 100, 99, 98, 97, 96 or 95. In some embodiments, the CTB polypeptide comprises a CTB fragment of the polypeptide of SEQ ID NO: 3 or 4 selected from: 2-95, 2-96, 2-97, 2-98, 2-99, 2-100, 2-101, 2-102, 2-103, 3-95, 3-96, 3-97, 3-98, 3-99, 3-100, 3-101, 3-102, 3-103, 4-95, 4-96, 4-97, 4-98, 4-99, 4-100, 4-101, 4-102, 4-103, 5-95, 5-96, 5-97, 5-98, 5-99, 5-100, 5-101, 5-102, 5-103, 6-95, 6-96, 6-97, 6-98, 6-99, 6-100, 6-101, 6-102, 6-103, 7-95, 7-96, 7-97, 7-98, 7-99, 7-100, 7-101, 7-102, 7-103

In some embodiments, the CTB polypeptide is a CTB polypeptide variant having one or more modifications to increase the expression of the polypeptide in a plant cell. In some embodiments, the CTB polypeptide is a CTB polypeptide variant selected from the CTB variants described in U.S. patent application Ser. No. 14/005,388, which is a 371 application of International Patent Application No. PCT/US12/29072, filed Mar. 14, 2012, which is incorporated herein by this reference.

In some embodiments, the CTB polypeptide can be a polypeptide having 85, 86, 87, 88, 89, 90, 91, 92, 93, 94, 95, 96, 97, 98, or 99% homology to the amino acid sequence of SEQ ID NO: 3, SEQ ID NO: 4, or a functional fragment thereof.

In some embodiments, the PV L2 fragment and the CTB polypeptide can be provided in a fusion protein. As used herein, “fusion protein” refers to a protein product of two or more genes or nucleotide sequences of interest that have been joined (e.g., covalently joined).

The Fusion of the L2 epitope to either the carboxy- or amino-terminus of Cholera toxin B subunit (CTB).

The CTB polypeptide can be provided upstream of the PV polypeptide fragment or downstream of the PV polypeptide fragment in the fusion protein. In some embodiments, the CTB polypeptide is provided at the carboxy-terminus of the fusion protein. In some embodiments, the CTB polypeptide is provided at the amino-terminus of the fusion protein.

As used herein with reference to an amino acid sequence, or a reference residue of an amino acid sequence, “upstream” refers to the amino acids closer to the amino-terminus of the amino acid sequence. Because the convention for presenting amino acid sequences is to present the sequence with the amino terminus to the left, writing the sequence from amino-terminus to carboxy-terminus, “upstream” refers to the amino acids to the left of a reference residue.

As used herein with reference to an amino acid sequence, or a reference residue of an amino acid sequence, “downstream” refers to the amino acids closer to the carboxy-terminus of the amino acid sequence. Because the convention for presenting amino acid sequences is to present the sequence with the carboxy-terminus to the right, writing the sequence from amino-terminus to carboxy-terminus, “downstream” refers to the amino acids to the right of a reference residue.

In should be recognized that in some embodiments, the fusion proteins can include multiple PV polypeptide fragment units and/or multiple CTB polypeptide units. In some embodiments, the fusion protein can include a first and a second PV polypeptide fragment, wherein the second PV polypeptide is of a different type than the first PV polypeptide fragment. For example, the first PV polypeptide fragment could be an HPV-16 polypeptide fragment and the second PV polypeptide fragment could be an HPV-18 polypeptide fragment.

Optionally, the fusion protein can further include additional amino acids, linker groups, polypeptides, or polypeptide fragments. For example, an additional polypeptide could be provided in the fusion protein to enhances desirable properties of the PV L2 polypeptide fragment, including for example antigenicity, cross-neutralizing responses, purification, bioavailability, etc. For example, in some embodiments a linker, comprising 1, 2, 3, 4, 5, 6, 7, 8, 9, or 10 amino acids is disposed between the PV L2 polypeptide and the CTB polypeptide. By way of a non-limiting example, in some embodiments including an HPV-16 L2 fragment and a CTB polypeptide, the linker can include two amino acids, AG.

In some embodiments, the polypeptide can further include one or more histidines. In some embodiments, the one or more histidines are provided upstream of the PV polypeptide fragment in the fusion protein. In some embodiments, the one or more histidines are provided downstream of the PV polypeptide fragment in the fusion protein. In some embodiments, the one or more histidines are provided at the carboxy-terminus of the fusion protein. In some embodiments, the one or more histidines are provided at the amino-terminus of the fusion protein. In some embodiments, the polypeptide includes a linker comprising 1, 2, 3, 4, 5, 6, 7, 8, 9, or 10 amino acids is disposed between the PV L2 polypeptide and the histidines.

Desired fusion proteins can be produced using recombinant technologies well known to those of ordinary skill in the art.

In some embodiments, the polypeptide has an affinity for GM1 ganglioside as determined by surface plasmon resonance (SPR). For example, the affinity (K_{D}) of the polypeptide for GM1 ganglioside as determined by surface plasmon resonance (SPR) can be less than 1 μM. In some embodiments, the affinity (K_{D}) of the polypeptide for GM1 ganglioside as determined by surface plasmon resonance (SPR) is greater than about 40, 50, 60, 70, 80, 90 100, 110, 120, 130, 140, 150, 160, 170, 180, 190, 200, 210, 220, 230, 240, 250, 260, 270, 280, 290, or 300 nM.

Has noted herein, the presently-disclosed subject matter includes compositions comprising a polypeptide as disclosed herein. In some embodiments, the composition is a pharmaceutical composition comprising a polypeptide of the presently-disclosed subject matter and a pharmaceutically-acceptable vehicle, carrier, or excipient. Suitable vehicles, carriers or excipients will be apparent to those of ordinary skill in the art upon study of this application. In some embodiments, the pharmaceutical composition can include an adjuvant. Suitable adjuvants will be apparent to those of ordinary skill in the art upon study of this application.

Compositions of the presently-disclosed subject matter can further include multiple distinct polypeptides as disclosed herein. In some embodiments, first and second polypeptides are distinct. For example, a first polypeptide can includes the CTB polypeptide provided upstream of the PV polypeptide fragment in a fusion protein, and the second polypeptide includes the CTB polypeptide provided downstream of the PV polypeptide fragment. For another example, the PV polypeptide fragment of the first polypeptide could be of a first type (e.g., HPV-16) and the PV polypeptide fragment of the second polypeptide could be of a second type (e.g., HPV-18).

The presently-disclosed subject matter further includes isolated nucleic acid molecules, including cDNA molecules, which encode the polypeptides of the presently-disclosed subject matter. The presently-disclosed subject matter further includes expression vectors comprising such nucleic acid sequences, operably linked to an expression cassette. With regard to the expression vector, in some embodiments, it is a tobacco mosaic virus (TMV)-based DNA plasmid. The presently-disclosed subject matter further includes plant cells transfected with such expression vector, or progeny thereof, wherein the cell or the progeny expresses the polypeptide. The plant cell can be, for example, a Nicotiana plant cell or a Nicotiana benthamiana plant cell.

The presently-disclosed subject matter further includes methods of producing a polypeptide as disclosed herein, which involve expressing the polypeptide in a plant cell and purifying the polypeptide. The presently-disclosed subject matter further includes methods of isolating a polypeptide from a plant tissue, which involve obtaining a plant cell expressing the polypeptide, extracting the polypeptide from the plant cell, and purifying the polypeptide from the plant cell. The plant cell can be, for example, a Nicotiana plant cell or a Nicotiana benthamiana plant cell.

In some embodiments particular embodiments of the presently-disclosed subject matter, a method of producing a fusion protein comprising a PV L2 polypeptide fused with a Cholera toxin B subunit (CTB) is provided. In some embodiments, the method includes: identifying a PV L2 polypeptide of interest and a CTB of interest; generating an expression vector comprising a gene encoding the PV L2 polypeptide and the CTB together as a fusion protein; transcribing the gene; introducing the transcribed gene into at least one cell (e.g., at least one eukaryotic cell); expressing the fusion protein from the transcribed gene within the cell; and isolating the fusion protein from the eukaryotic cell.

With regard to the step of transcribing the gene, in some embodiments, the gene is under the control of a regulatory element. In some embodiments, the regulatory element can be a promoter, e.g., a T7 promoter. In some embodiments, the transcription includes in vitro transcription using T7 polymerase.

With regard to the step of introducing the transcribed gene into a eukaryotic cell, in some embodiments, the transcribed gene is introduced by infecting the eukaryotic cell with the transcribed gene, which can be an infectious RNA polynucleotide. In some embodiments, the eukaryotic cell is a Nicotiana benthamiana cell. In some embodiments, the eukaryotic cell is a plurality of Nicotiana benthamiana cells. In some embodiments, the plurality of Nicotiana benthamiana cells is a Nicotiana benthamiana seedling.

The presently-disclosed subject matter further includes methods of treating a papillomavirus (PV) infection in a subject. In some embodiments, the method includes administering an effective amount of a composition comprising a PV L2 polypeptide and a second polypeptide, as described above. In some embodiments, the method includes administering the composition by contacting the composition to a mucosa of a subject.

As used herein, the terms “treatment” or “treating” relate to any treatment of a PV infection, including but not limited to prophylactic treatment and therapeutic treatment. As such, the terms treatment, treating, affecting treatment, and being effective for treatment include, but are not limited to: conferring protection against a PV infection; preventing a PV infection; reducing the risk of PV infection; ameliorating or relieving symptoms of a PV infection; eliciting an immune response against a PV or an antigenic component thereof; inhibiting the development or progression of a PV infection; inhibiting or preventing the onset of symptoms associated with a PV infection; reducing the severity of a PV infection; and causing a regression of a PV infection or one or more of the symptoms associated with a PV infection.

As used herein, the term “PV infection” refers to a colonization of a cell of a subject by a papillomavirus (PV). In some embodiments, infection refers to a colonization of a cell of the subject and an interference with normal functioning of the cell. The interference with normal functioning of the cell of the subject can result in the onset, and ultimately the expression, of symptoms in the subject.

Symptoms associated with PV infection are known to those of ordinary skill in the art and can include, but are not limited to: formation of papillomas or warts, which in infants and young children can develop into RRP; development of precancerous lesions; and development of cancer. The presence of an infection can be assessed using methods known to those of ordinary skill in the art. In some cases, the presence of a PV infection can be determined by detecting HPV DNA or RNA in a sample obtained from the subject. In some cases, the presence of a PV infection can be determined using antibodies to HPV capsid proteins, or using virus-like particles to detect serum antibodies. In some cases, non-structural proteins can be useful for detection of existing infections, and an immunoassay for the viral non-structural proteins could be useful for detection of infections in tissue samples, using techniques known to those of ordinary skill in the art, such as immunohistochemistry, western blot, or ELISA. In some cases, the presence of a PV infection can be determined by identifying a symptom associated with PV infection.

The presently-disclosed subject matter further includes methods of eliciting an immune response in a subject, which involve administering to a subject in need thereof an effective amount of a polypeptide or composition as disclosed herein. In some embodiments, administering an effective amount of the polypeptide increases an amount of IgG, IgA, IgM, effector T cells, regulatory T cells, or combinations thereof in a subject. In some embodiments, the polypeptide is administered to a mucosa of the subject. In some embodiments, the polypeptide is administered sublingually to the subject. In some embodiments, sublingual administration results in increased production of HPV-16 and HPV-18 cross-neutralizing antibodies as compared to subcutaneous administration.

As used herein, “immunizing” and “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response can be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade and/or activation of complement) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “effective amount” refers to a dosage or a series of dosages sufficient to affect treatment for a PV infection in a subject. This can vary depending on the subject, the PV (e.g., PV-type) and the particular treatment being affected. The exact amount that is required will vary from subject to subject, depending on the species, age, and general condition of the subject, the particular adjuvant being used, administration protocol, and the like. As such, the effective amount will vary based on the particular circumstances, and an appropriate effective amount can be determined in a particular case by one of ordinary skill in the art using only routine experimentation.

Administration protocols can be optimized using procedures generally known in the art. A single dose can be administered to a subject, or alternatively, two or more inoculations can take place with intervals of several weeks to several months. The extent and nature of the immune responses induced in the subject can be assessed using a variety of techniques generally known in the art. For example, sera can be collected from the subject and tested, for example, for PV DNA or RNA in a sera sample, detecting the presence of antibodies to PV or antigenic fragments thereof using, for example, PV VLPs, or monitoring a symptom associated with PV infection. Relevant techniques are well described in the art, e.g., Coligan et al. Current Protocols in Immunology, John Wiley & Sons Inc. (1994), which is incorporated herein by this reference.

As used herein, the term subject refers to both human and animal subjects. Thus, veterinary therapeutic uses are provided in accordance with the presently-disclosed subject matter. A subject susceptible to an HPV infection can be a human subject.

While the terms used herein are believed to be well understood by one of ordinary skill in the art, definitions are set forth herein to facilitate explanation of the presently-disclosed subject matter.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Although any methods and materials similar or equivalent to those described herein can be used in the practice or testing of the presently-disclosed subject matter, representative methods and materials are described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

The presently-disclosed subject matter is further illustrated by the following specific but non-limiting examples. The following examples may include compilations of data that are representative of data gathered at various times during the course of development and experimentation related to the present invention.

### EXAMPLES

By way of an introduction to the studies described herein, there are over 40 different human papillomavirus (HPV) types, including at least 15 high-risk oncogenic strains of HPV that have been associated with the development of anogenital and oropharyngeal cancers. HPV infection is attributed to multiple cancer types (e.g., cervical (100%), oropharyngeal (63%), anal (93%), penile (40%), vulvar (51%), and vaginal (64%)). HPV types 16 and 18 have been linked to 70% of cervical carcinomas.

Current HPV vaccines are primarily type-specific, consisting of virus-like particles containing the L1 major capsid protein. These vaccines induce type-specific neutralizing antibodies against oncogenic HPV types 16, 18 and closely related strains. Among the goals of the research described herein was the development of a mucosally-deliverable HPV vaccine based on L2 minor capsid protein, capable of inducing production of broadly cross-neutralizing antibodies from a single antigen.

The present inventors contemplated that a polypeptide including fusion of an L2 epitope of an HPV to either the carboxy- or amino-terminus of a Cholera toxin B subunit (CTB) would more-efficiently present the RG-1 epitope and induce a greater cross-neutralizing immune response in a subject. Delivery of CTB via mucosal routes was contemplated by the present inventors to provide adequate immune response to cross-neutralize HPV.

### Example 1

With reference to FIG. 1C, amino acids 8-37 of HPV 16 L2 contains the RG-1 epitope and was fused to the N-terminal or C-terminal of CTB. A 6×His tag was added to the same terminus as the HPV epitope to aid in purification. CTB-L2 fusion proteins were expressed in Nicotiana benthamiana using the MagnICON® vector system. Fusion proteins were purified by immobilized metal affinity chromatography (IMAC) and ceramic hydroxyapatite, in accordance with the procedures described in Hamorsky, et al., “Efficient Single Tobamoviral Vector-based Bioproduction of Broadly Neutralizing anti-HIV-1 Monoclonal Antibody VRC01 in Nicotiana Plants and Its Utility in Combination Microbicides,” Antimicrobial Agents and Chemotherapy (2013), which is incorporated herein by this reference. For purposes of the example, and without wishing to be bound by theory, for ease of expression, purification, and/or convenience, the antigen sequences included the following: His-6 and H13R mutation in the CTB polypeptide sequence to allow for purification through the His tag, because H13 forms a His patch in the CTB pentamer. Rice alpha amylase is to direct expression through the secretory pathway, which facilitates disulfide bond formation in the RG-1 epitope, facilitating expression. G between the rice alpha amylase and the His-6 to improve signal peptide cleavage and facilitate expression. GGGS, which is a flexible linker, facilitating expression. AG linker, which is a less flexible, more stable liker, facilitating expression. SEKDEL, facilitating ER retention, expression.

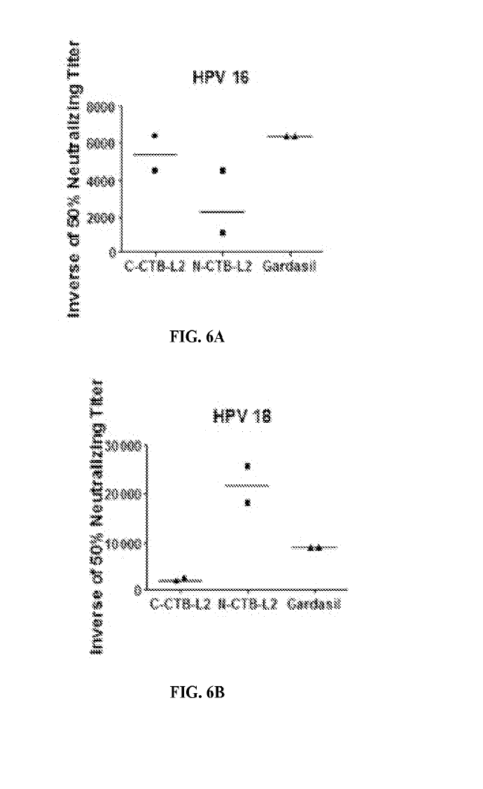

With reference to FIG. 2, Antigen stability was assessed through western blots and SDS-PAGE to confirm the presence of CTB and the L2 epitope.

A series of cross-neutralization studies were conducted as follows. Each rabbit was immunized 3 times with 100 μg of antigen. The initial immunization consisted of Complete Freunds adjuvant and subsequent injections Incomplete Freunds adjuvant.
Day 0 Treatment 1
Day 14 Treatment 2
Day 28 Treatment 3
Day 35 Serum Collection
   Serum was collected and analyzed for CTB and L2 antibody concentration by ELISA and capacity for cross-neutralization by HPV pseudovirus neutralization assay.

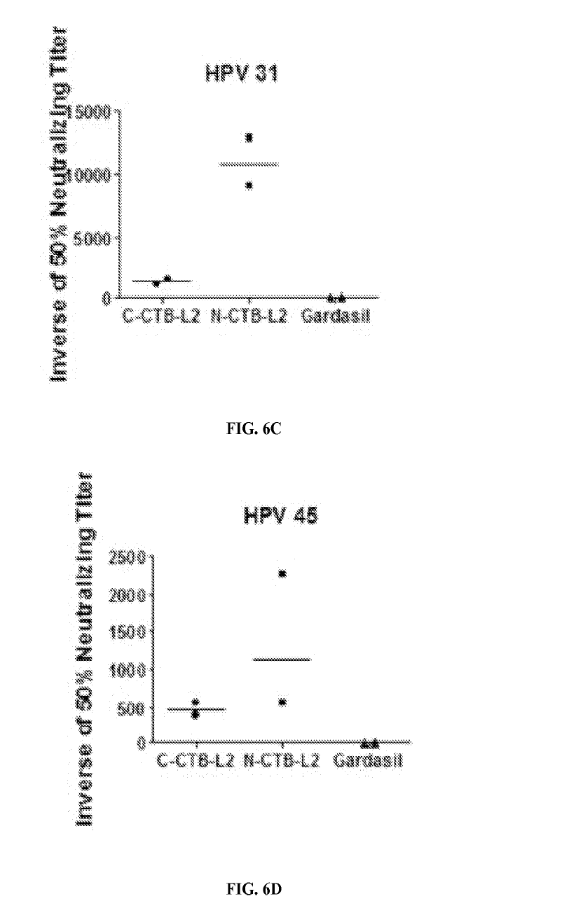

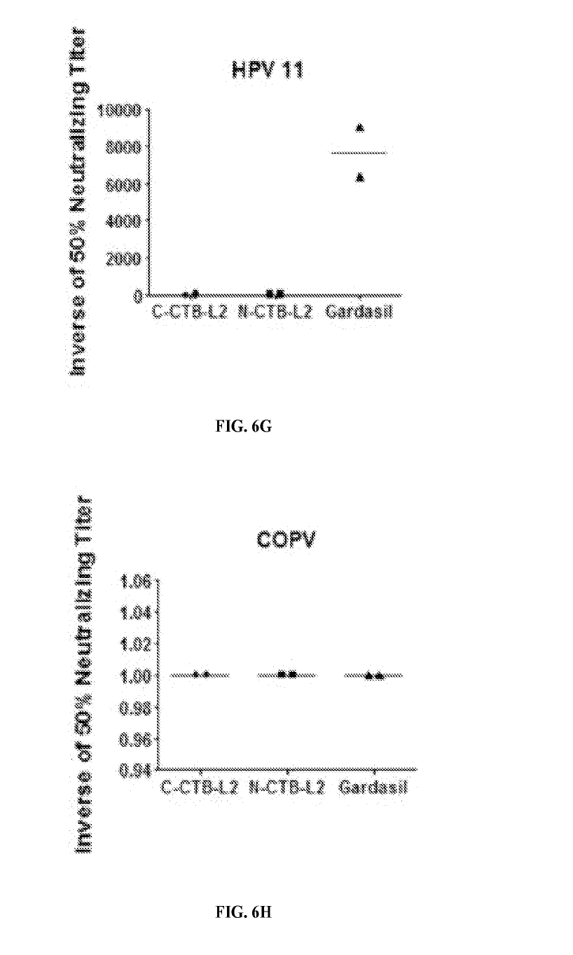

With reference to FIG. 3, and with further reference to FIG. 5, neutralization titers against HPV-16, 18, 31, and 45 were assessed in serum from two rabbits inoculated with C-CTB-L2 and N-CTB-L2. The inverse of the final dilution exhibiting 50% virus inhibition was averaged between animals. Serum from animals vaccinated with N-CTB-L2 appears to cross-neutralize virus more effectively. Antibodies reactive against L2 were assessed by ELISA using a BSA conjugated peptide homologous to the RG-1 region of HPV-16 L2 immobilized on the plate. Endpoint titers were determined to be the last dilution with a signal above 2× the background signal.

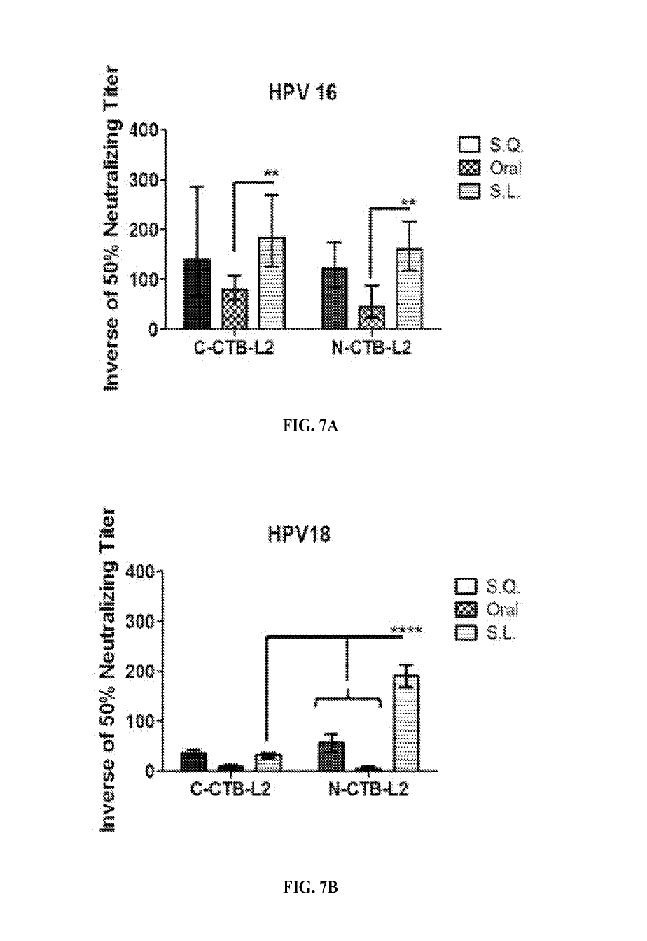

With reference to FIG. 7, neutralization titers were averaged across each group and analyzed by a one-way ANOVA with Bonferroni's post-hoc tests. No significant differences were observed in HPV 16 neutralizations of C-CTB-L2 or N-CTB-L2 vaccinated animals when comparing the same route of administration across vaccine candidates. Significant differences were observed when comparing HPV 16 neutralizations across modes of delivery within each treatment group. Oral delivery produced significantly less neutralizing antibodies when compared to sublingual delivery for both constructs. Serum from animals inoculated with N-CTB-L2 demonstrated significantly greater cross-neutralization of HPV 18 when delivered sublingually relative to C-CTB-L2 delivered sublingually or N-CTB-L2 delivered orally or subcutaneously. n=5 ** p-value <0.01., **** p-value <0.0001.

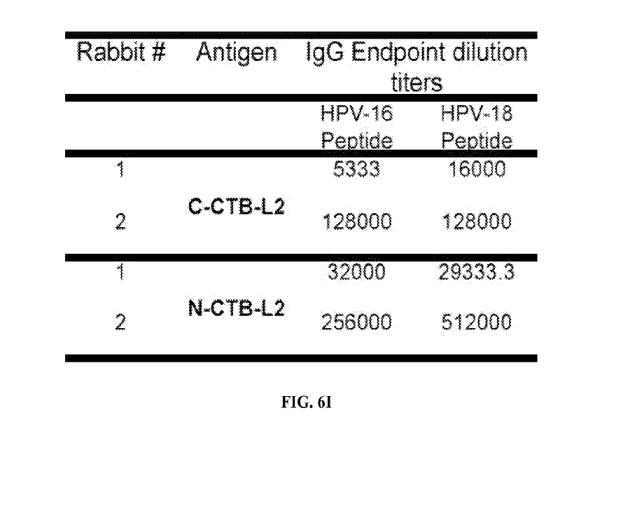

With reference to FIGS. 6A-6I, in a subsequent study, neutralization titers against HPV-16, 18, 31, 45, 58, 6, and 11, and COPV were assessed in serum from two rabbits inoculated with C-CTB-L2 two rabbits inoculated with N-CTB-L2, and two rabbits inoculated with Gardasil® (L1 VLP quatravalent HPV-16, 18, 6, and 11).

Both antigens induce the production of cross-neutralizing antibodies against HPV-16, 18, 31, 45, 58, and 6. Serum from animals vaccinated with N-CTB-L2 appears to cross-neutralize virus most effectively. Without wishing to be bound by theory, it appears that the amino-terminal fusion of the L2 epitope to CTB more efficiently presents the RG-1 epitope for production of cross-neutralizing antibodies.

### Example 2

With reference to FIG. 1C, amino acids 8-37 of HPV 16 L2 contains the RG-1 epitope and was fused to the N-terminal or C-terminal of CTB. A 6×His tag was added to the same terminus as the HPV epitope to aid in purification. CTB-L2 fusion proteins were expressed in Nicotiana benthamiana. Fusion proteins were purified in accordance with the procedures described in Hamorsky, et al., Antimicrobial Agents and Chemotherapy (2013).

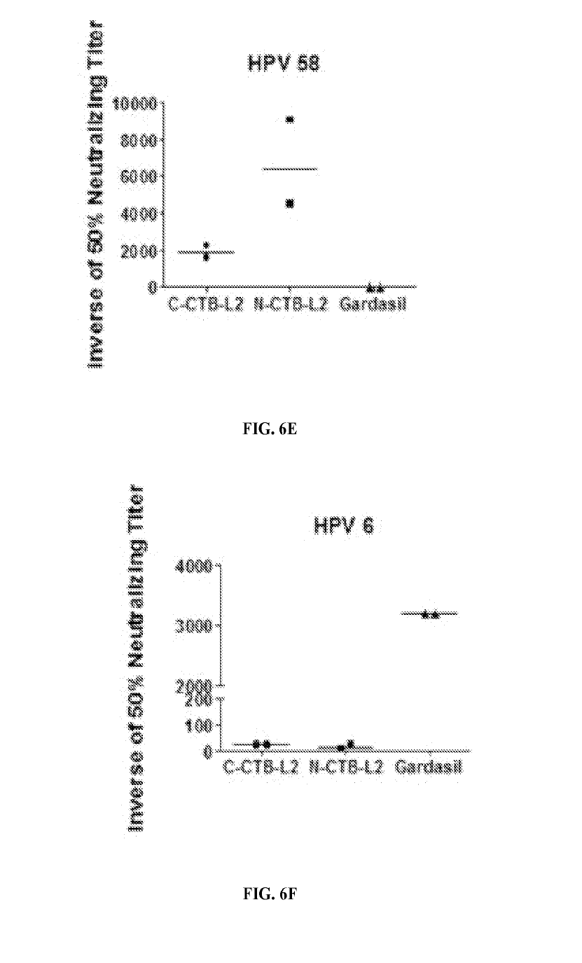

With reference to FIG. 4, Antigen stability was assessed to confirm the presence of CTB and the L2 epitope.

Surface Plasmon Resonance (SPR) was used to evaluate the affinity of N-CTB-L2 and C-CTB-L2 (K_{D}) for GM1 relative to CTB. SPR uses electromagnetic waves that propagate along the boundary between a metal and a dielectric medium (air or gas) stimulated by light. Light is absorbed at particular wavelengths based on the resonance of surface plasmons. Changes in the surface alters surface plasmons and changes the refractive index. SPR methods are further described in Hamorsky et al., “Rapid and scalable plant-based production of a cholera toxin B subunit variant to aid in mass vaccination against cholera outbreaks,” PLoS Negl Trop Dis. 2013; 7(3):e2046, which is incorporated herein by this reference.

GM1 ganglioside is a cell membrane lipid with the oligosaccharide portion exposed to the extracellular space and is ubiquitously expressed on cells. GM1 ganglioside binding by CTB is necessary for immunomodulatory function. As shown in the following table, the K_{D} of N-CTB-L2 and C-CTB-L2 for GM1 relative to CTB was determined to be higher, but was maintained within the nM range understood to be necessary for immune activation.

| Construct | K_{D} (nM) |
|---|---|
| CTB | 39.10 ± 3.95 |
| N-CTB-L2 | 188.2 ± 8.97 |
| C-CTB-L2 | 65.72 ± 4.80 |

Analyzed by a one-way ANOVA with Bonferroni's post-hoc tests: N-CTB-L2 vs. CTB - p < 0.0001, C-CTB-L2 vs. CTB - p < 0.001, N-CTB-L2 vs. C-CTB-L2 - p < 0.0001

Interactions of CTB-L2 with the surface of cells through GM-1 binding was investigated through double fluorescence of CTB and the L2 region while competing with soluble GM-1. 20 μg/mL of CTB, N-CTB-L2, or C-CTB-L2 was incubated with multiple concentrations of GM-1 overnight at 4° C. RAW 264.7 cells, a murine macrophage cell-line, were treated overnight at 37° C. with antigen GM-1 mixture. Cells were fixed and treated with antibodies for L2 and CTB and the fluorescence measured by FACS.

With reference to FIGS. 6A and 6B, CTB has a higher level of binding to the cells and requires greater concentrations of soluble GM-1 to inhibit binding than either the C- or N-terminal fusion. These data are consistent with the higher affinity of CTB shown by SPR, but also demonstrate that N-CTB-L2 and C-CTB-L2 can bind to immune effector cells through GM-1. N-CTB-L2 has a higher level of binding of the L2 respective antibody the C-CTB-L2, which may be related to an increased exposure upon binding and the reason for the improved immunogenicity.

### Example 3

With reference to FIG. 1C, amino acids 8-37 of HPV 16 L2 contains the RG-1 epitope and was fused to the N-terminal or C-terminal of CTB. A 6×His tag was added to the same terminus as the HPV epitope to aid in purification. CTB-L2 fusion proteins were expressed in Nicotiana benthamiana. Fusion proteins were purified in accordance with the procedures described in Hamorsky, et al., Antimicrobial Agents and Chemotherapy (2013).

With reference to FIG. 4, Antigen stability was assessed to confirm the presence of CTB and the L2 epitope.

A mouse mucosal immunization trial was conducted. DMPA (Depot medroxyprogesterone acetate) was used to synchronize the estrus cycle of the mice. Each treatment (N-CTB-L2 or C-CTB-L2) consisted of a 20 μg dose of antigen diluted in PBS. Immunization Routes included: Oral—100 μL volumes with a sodium bicarbonate solution; Sublingual—5 μL volumes; and Subcutaneous—100 μL volumes. Five animals were provided per immunization route per antigen. The following treatment regimen was used.

| | |
|---|---|
| Day 0 | Treatment 1 |
| Day 7 | Treatment 2 |
| Day 21 | Treatment 3 |
| Day 28 | Treatment 4 |
| Day 35 | Treatment 5 |
| Day 42 | Serum Collection |

With reference to FIGS. 7A and 7B, delivery of CTB-L2 via mucosal routes provides adequate immune response to produce broadly cross-neutralizing HPV antibodies. Sublingual delivery of antigen induces a significantly greater viral neutralization response relative to oral delivery and provides the best cross-neutralization response when used to deliver N-CTB-L2.

An additional mouse mucosal immunization trial was conducted, using the treatment regimen outlined above. Immunization Routes included: Sublingual—5 μL volumes, and Subcutaneous—100 μL volumes. Serum and lavage samples pooled from 16 animals with 4 animals per group/immunization route (n=4) to allow for analysis in connection with additional HPV types.

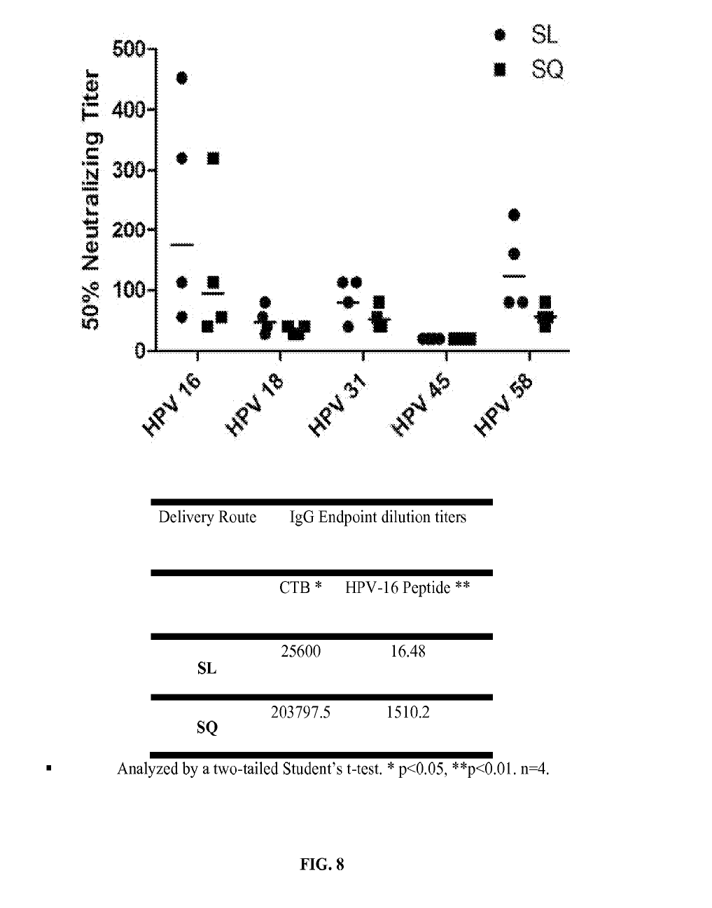

With reference to FIG. 8, delivery of N-CTB-L2 sublingually provides an immune response equivalent or better than subcutaneous delivery and produce broadly cross-neutralizing HPV antibodies. The IgG antibodies produced after sublingual delivery are significantly fewer than after subcutaneous delivery implying the protective antibody type is of a different Ig class, potentially IgA. As one of ordinary skill in the art will recognize this is a surprising and unexpected finding.

The forgoing examples indicate the following. CTB-L2 fusion proteins expressed in plants offer the potential of a less expensive HPV prophylactic vaccine which offers a broader spectrum of protection against oncogenic strains of the HPV. Delivery of CTB-L2 via mucosal routes provides adequate immune response to produce broadly cross-neutralizing HPV antibodies. Both antigens induced the production of cross-neutralizing antibodies against HPV-16, 18, 31, and 45. The amino-terminus fusion of the L2 epitope to CTB more efficiently presents the RG-1 epitope for production of cross-neutralizing antibodies. Sublingual delivery of antigen induces a significantly greater viral neutralization response relative to oral delivery and provides the best cross-neutralization response when used to deliver N-CTB-L2.

All publications, patents, and patent applications mentioned in this specification, including those set forth in the following list, are herein incorporated by reference to the same extent as if each individual publication, patent, or patent application was specifically and individually indicated to be incorporated by reference.

### REFERENCES

1) Garcea R L, and DiMaio D. The Papillomaviruses. New York: Springer, 2007. Print.
2) McKenzie S J, and Halsey, J F. “Cholera Toxin B subunit as a carrier protein to stimulate a mucosal immune response.” J. Immunology (1984) 133(4):1818-24.
3) Rubio I, et al. “The N-terminal region of the human papillomavirus L2 protein contains overlapping binding sites for neutralizing, cross-neutralizing and non-neutralizing antibodies.” Virology (2011) 409:348-359.
4) Subhashini J, et al. “Vaccination with multimeric L2 fusion protein and L1 VLP or capsomeres to broaden protection against HPV infection.” Vaccine (2010) 28:4478-4486.
5) Van Ranst M, et al. “Phylogenetic classification of human papillomaviruses: correlation with clinical manifestations.” J. Gen. Virology (1992) pt10: 2653-60.
6) Hamorsky, et al., “Efficient Single Tobamoviral Vector-based Bioproduction of Broadly Neutralizing anti-HIV-1 Monoclonal Antibody VRC01 in Nicotiana Plants and Its Utility in Combination Microbicides,” Antimicrobial Agents and Chemotherapy (2013).
7) Hamorsky et al., “Rapid and scalable plant-based production of a cholera toxin B subunit variant to aid in mass vaccination against cholera outbreaks,” PLoS Negl Trop Dis. 2013; 7(3):e2046

It will be understood that various details of the presently disclosed subject matter can be changed without departing from the scope of the subject matter disclosed herein. Furthermore, the foregoing description is for the purpose of illustration only, and not for the purpose of limitation.

SEQUENCES

SEQ ID NO: 1-HPV-16 L2 Amino Acid
MQVTFIYILVITCYENDVNVYHIFFQMSLWLPSEATVYLPPVPVSKVVSTDEYVARTNIYYHAGTSRLLAV
GHPYFPIKKPNNNKILVPKVSGLQYRVFRIHLPDPNKFGFPDTSFYNPDTQRLVWACVGVEVGRGQPLGVG
ISGHPLLNKLDDTENASAYAANAGVDNRECISMDYKQTQLCLIGCKPPIGEHWGKGSPCTNVAVNPGDCPPL
ELINTVIQDGDMVHTGFGAMDFTTLQANKSEVPLDICTSICKYPDYIKMVSEPYGDSLFFYLRREQMFVRHL
FNRAGTVGENVPDDLYIKGSGSTANLASSNYFPTPSGSMVTSDAQIFNKPYWLQRAQGHNNGICWGNQLFVT
VVDTTRSTNMSLCAAISTSETTYKNTNFKEYLRHGEEYDLQFIFQLCKITLTADVMTYIHSMNSTILEDWNFG
LQPPPGGTLEDTYRFVTQAIACQKHTPPAPKEDDPLKKYTFWEVNLKEKFSADLDQFPLGRKFLLQAGLKAKP
KFTLGKRKATPTTSSTSTTAKRKKRKL
SEQ ID NO: 2-HPV-16 L2 Nucleotide
ACCESSION NC 001526; VERSION NC_001526.2 GI: 310698439
ATGCAGGTGACTTTTATTTACATCCTAGTTATTACATGTTACGAAAACGACGTAAACGTTTACCATATTT
TTTTTCAGATGTCTCTTTGGCTGCCTAGTGAGGCCACTGTCTACTTGCCTCCTGTCCCAGTATCTAAGGT
TGTAAGCACGGATGAATATGTTGCACGCACAAACATATATTATCATGCAGGAACATCCAGACTACTTGCA
GTTGGACATCCCTATTTTCCTATTAAAAAACCTAACAATAACAAAATATTAGTTCCTAAAGTATCAGGAT
TACAATACAGGGTATTTAGAATACATTTACCTGACCCCAATAAGTTTGGTTTTCCTGACACCTCATTTTA
TAATCCAGATACACAGCGGCTGGTTTGGGCCTGTGTAGGTGTTGAGGTAGGTCGTGGTCAGCCATTAGGT
GTGGGCATTAGTGGCCATCCTTTATTAAATAAATTGGATGACACAGAAAATGCTAGTGCTTATGCAGCAA
ATGCAGGTGTGGATAATAGAGAATGTATATCTATGGATTACAAACAAACACAATTGTGTTTAATTGGTTG
CAAACCACCTATAGGGGAACACTGGGGCAAAGGATCCCCATGTACCAATGTTGCAGTAAATCCAGGTGAT
TGTCCACCATTAGAGTTAATAAACACAGTTATTCAGGATGGTGATATGGTTCATACTGGCTTTGGTGCTA
TGGACTTTACTACATTACAGGCTAACAAAAGTGAAGTTCCACTGGATATTTGTACATCTATTTGCAAATA
TCCAGATTATATTAAAATGGTGTCAGAACCATATGGCGACAGCTTATTTTTTTATTTACGAAGGGAACAA
ATGTTTGTTAGACATTTATTTAATAGGGCTGGTACTGTTGGTGAAAATGTACCAGACGATTTATACATTA
AAGGCTCTGGGTCTACTGCAAATTTAGCCAGTTCAAATTATTTTCCTACACCTAGTGGTTCTATGGTTAC
CTCTGATGCCCAAATATTCAATAAACCTTATTGGTTACAACGAGCACAGGGCCACAATAATGGCATTTGT
TGGGGTAACCAACTATTTGTTACTGTTGTTGATACTACACGCAGTACAAATATGTCATTATGTGCTGCCA
TATCTACTTCAGAAACTACATATAAAAATACTAACTTTAAGGAGTACCTACGACATGGGGAGGAATATGA
TTTACAGTTTATTTTTCAACTGTGCAAAATAACCTTAACTGCAGACGTTATGACATACATACATTCTATG
AATTCCACTATTTTGGAGGACTGGAATTTTGGTCTACAACCTCCCCCAGGAGGCACACTAGAAGATACTT
ATAGGTTTGTAACCCAGGCAATTGCTTGTCAAAAACATACACCTCCAGCACCTAAAGAAGATGATCCCCT
TAAAAAATACACTTTTTGGGAAGTAAATTTAAAGGAAAAGTTTTCTGCAGACCTAGATCAGTTTCCTTTA
GGACGCAAATTTTTACTACAAGCAGGATTGAAGGCCAAACCAAAATTTACATTAGGAAAACGAAAAGCTA
CACCCACCACCTCATCTACCTCTACAACTGCTAAACGCAAAAAACGTAAGCTGTAA
SEQ ID NO: 3-CTB Amino Acid
TPQNITDLCA EYHNTQIHTL NDKIFSYTES LAGKREMAII TFKNGATFQV EVPGSQHIDS
QKKAIERMKD TLRIAYLTEA KVEKLCVWNN KTPHAIAAIS MAN
SEQ ID NO: 4-CTB (His 13 Arg) Amino Acid
TPQNITDLCA EYRNTQIHTL NDKIFSYTES LAGKREMAII TFKNGATFQV EVPGSQHIDS
QKKAIERMKD TLRIAYLTEA KVEKLCVWNN KTPHAIAAIS MAN
SEQ ID NO: 5-CTB (His 13 Arg) Nucleotide
Accccacaaaacatcactgacttgtgtgctgagtacagaaacacccaaatccacaccctcaatgacaagatctttag
ctacaccgagagccttgctggcaagagggagatggctatcatcaccttcaagaatggtgctaccttccaagtggagg
tgcctggaagccaacacattgatagccaaaagaaggccattgagaggatgaaggacacacttaggatagcttacctc
actgaggctaaggtggagaagctttgtgtgtggaacaacaagaccccccatgctattgctgcaatcagcatggccaac
SEQ ID NO: 6-N-CTB-L2 Amino Acid

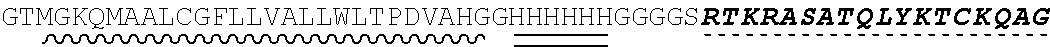

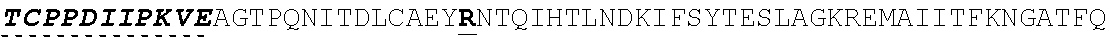

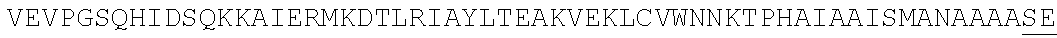

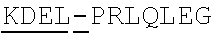

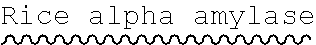

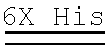

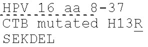

SEQ ID NO: 7-N-CTB-L2 Nucleotide
ggcaccatggggaagcaaatggccgccctgtgtggctttctcctcgtggcgttgctctggctcacgcccgacgtcgc
gcatggtgggcaccaccatcaccatcatggaggtggcggctcacgtacaaaaagagctagtgccactcaactttaca
aaacatgcaagcaggctggtacttgcccgccagatattatccctaaagttgaagccggcaccccacaaaacatcact
gacttgtgtgctgagtacagaaacacccaaatccacaccctcaatgacaagatctttagctacaccgagagccttgc
tggcaagagggagatggctatcatcaccttcaagaatggtgctaccttccaagtggaggtgcctggaagccaacaca
ttgatagccaaaagaaggccattgagaggatgaaggacacacttaggatagcttacctcactgaggctaaggtggag
aagctttgtgtgtggaacaacaagaccccccatgctattgctgcaatcagcatggccaacgctgcggccgcatccga
gaaggatgaactctaacctaggctgcagctcgaggga
SEQ ID NO: 8-C-CTB-L2 Amino Acid

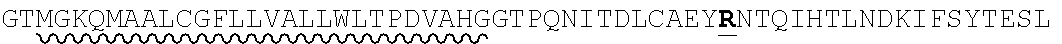

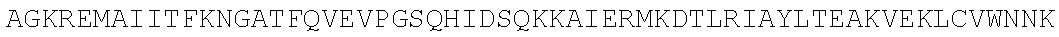

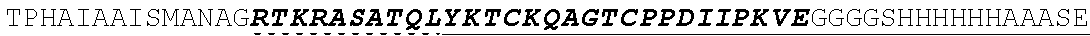

KDEL-PRLQLEG

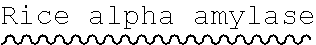

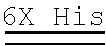

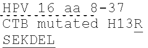

SEQ ID NO: 9-C-CTB-L2 Nucleotide
Ggcaccatggggaagcaaatggccgccctgtgtggctttctcctcgtggcgttgctctggctcacgcccgacgtcgc
gcatggtgggaccccacaaaacatcactgacttgtgtgctgagtacagaaacacccaaatccacaccctcaatgaca
agatctttagctacaccgagagccttgctggcaagagggagatggctatcatcaccttcaagaatggtgctaccttc
caagtggaggtgcctggaagccaacacattgatagccaaaagaaggccattgagaggatgaaggacacacttaggat
agcttacctcactgaggctaaggtggagaagctttgtgtgtggaacaacaagaccccccatgctattgctgcaatca
gcatggccaacgccggccgtacaaaaagagctagtgccactcaactttacaaaacatgcaagcaggctggtacttgc
ccgccagatattatccctaaagttgaaggaggtggcggctcacaccaccatcaccatcatgctgcggccgcatccga
gaaggatgaactctaacctaggctgcagctcgaggga

1. An isolated polypeptide, comprising a papillomavirus (PV) minor capsid (L2) polypeptide fragment and a cholera toxin B subunit (CTB) polypeptide.

2-4. (canceled)

5. The polypeptide of claim 1, wherein the PV L2 polypeptide has the sequence of a HPV L2 polypeptide fragment, and wherein the HPV L2 polypeptide fragment is from an HPV-type, selected from the group consisting of HPV-1, -2, -5, -6, -8, -6, -11, -16, -18, -26, -31, -33, -35, -39, -40, -45, -51, -52, -53, -56, -58, 59, -67, -68, -73, and -82.

6-9. (canceled)

10. The polypeptide of claim 1, wherein the PV L2 polypeptide fragment comprises a fragment extending from amino acid 5, 6, 7, 8, 9, 10, 11, 12, 13, 14, 15, 16, 17, or 18, and extending to amino acid 120, 115, 110, 105, 100, 95, 90, 85, 80, 75, 74, 73, 72, 71, 70, 69, 68, 67, 66, or 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, or 35 of the full-length PV L2 polypeptide.

11. The polypeptide of claim 1, where the PV L2 polypeptide fragment comprises a fragment extending from Furin cleavage and including the Kawana epitope.

12. The polypeptide of claim 1, where the PV L2 polypeptide fragment comprises a fragment selected from: 17-36, 8-37, or 13-70.

13-14. (canceled)

15. The polypeptide of claim 1, wherein the PV L2 polypeptide fragment is a polypeptide having 85, 86, 87, 88, 89, 90, 91, 92, 93, 94, 95, 96, 97, 98, or 99% homology to the amino acid sequence of a fragment of a wild type PV L2 polypeptide.

16. The polypeptide of claim 1, wherein the PV L2 polypeptide fragment is a polypeptide having 85, 86, 87, 88, 89, 90, 91, 92, 93, 94, 95, 96, 97, 98, or 99% homology to the amino acid sequence of a fragment of wild type HPV-16 L2 polypeptide.

17. The polypeptide of claim 5, wherein the HPV L2 polypeptide is from a first HPV-type, and the composition is effective for treatment of infections caused by the first HPV-type and at least one additional HPV-type.

18. The polypeptide of claim 1, wherein the CTB polypeptide is a wild type CTB or fragment thereof, or a CTB variant having one or more modifications to increase the expression of the polypeptide in a plant cell.

19. The polypeptide of claim 18, wherein the CTB polypeptide is a CTB fragment extending from amino acid 1, 2, 3, 4, 5, or 6 and extending to amino acid 95, 96, 97, 98, 99, 100, 101, 102, or 103 of the full-length CTB polypeptide.

20. The polypeptide of claim 18, wherein the CTB polypeptide fragment is a polypeptide having 85, 86, 87, 88, 89, 90, 91, 92, 93, 94, 95, 96, 97, 98, or 99% homology to the amino acid sequence of CTB polypeptide.

21. The polypeptide of claim 1, wherein the PV polypeptide fragment and the CTB polypeptide are provided in a fusion protein.

22-26. (canceled)

27. The polypeptide of claim 21, wherein the fusion protein further includes a second PV polypeptide fragment of a different type than the PV polypeptide fragment.

28. The polypeptide of claim 21, wherein PV polypeptide fragment is an HPV polypeptide fragment, and the fusion protein further includes a second HPV polypeptide fragment of a different type than the HPV polypeptide fragment.

29-31. (canceled)

32. The polypeptide of claim 21, and further comprising one or more histidines.

33-38. (canceled)

39. The polypeptide of claim 21, and further comprising a linker disposed between the CTB polypeptide and the PV polypeptide fragment.

40. The polypeptide of claim 39, wherein the linker comprises 1, 2, 3, 4, 5, or 6 amino acids.

41-42. (canceled)

43. The polypeptide of claim 1, comprising the amino acid sequence of any one of SEQ ID NO: 1, 3, 4, 6, and 8.

44-46. (canceled)

47. A pharmaceutical composition comprising the polypeptide of claim 1 and a pharmaceutically-acceptable vehicle, carrier, or excipient.

48. The pharmaceutical composition of claim 47, wherein the pharmaceutical composition further comprises an adjuvant.

49-52. (canceled)

53. A cDNA molecule, comprising a sequence that encodes the polypeptide of claim 1.

54-61. (canceled)

62. A method of treating a papillomavirus (PV) infection in a subject, comprising contacting the polypeptide of claim 1 to a mucosa of a subject.

63-67. (canceled)
